(19) United States
(12) Patent Application Publication
(10) Pub. No.: US 2015/0119679 A1
(43) Pub. Date: Apr. 30, 2015

(54) HEALTH MONITOR

(71) Applicants: INVENTEC CORPORATION, TAIPEI CITY (TW); Inventec (Pudong) Technology Corporation, Shanghai (CN)

(72) Inventor: Shih-Jung HUANG, TAIPEI CITY (TW)

(73) Assignees: INVENTEC CORPORATION, TAIPEI CITY (TW); Inventec (Pudong) Technology Corporation, Shanghai (CN)

(21) Appl. No.: 14/201,920

(22) Filed: Mar. 9, 2014

(30) Foreign Application Priority Data
Oct. 28, 2013 (CN) 201310521416.3

(51) Int. Cl. A61B 5/107; A61B 5/00

(52) U.S. Cl. CPC A61B 5/1072; A61B 5/6804; A61B 5/746
USPC 600/407; 600/587; 600/586; 340/686.1

(57) ABSTRACT

A health monitor includes a belt, a belt-buckle and a reader. The belt-buckle is connected to an end of the belt, and is configured to buckle up with the belt when the belt is rounded in a form of a ring. The reader is located at the belt-buckle, and is configured to read a buckling position where the belt-buckle and the belt buckle up.

### RELATED APPLICATIONS

This application claims priority to Chinese Application Serial Number 201310521416.3, filed Oct. 28, 2013, which is herein incorporated by reference.

### BACKGROUND

1. Field of Invention

The present invention generally relates to a health monitor. More particularly, the present invention relates to a health monitor monitoring the change of body shape.

2. Description of Related Art

Modern medicine has proven that obesity relates to various chronic diseases, such as diabetes, heart disease, hypertension, stroke and certain cancers. However, modern people live in a less dynamic style, and, In office, they often sit in front of a computer desk working for all day long, and, at home, huddle on the sofa watching television. With less physical exercises and more refined and high-calorie foods, people are apt to be overweighed unconsciously. As the prevailing trend of obesity, a variety of corresponding diseases occur more and more frequently. Many countries increasingly suffer from heavy medical burden, which directly affects the economic development of the countries.

As a result, monitoring the change of personal body shape has become as a must in a society, and paying constant attention to the change of waistline has become an important index of health management.

### SUMMARY

A technical aspect of the present invention provides a health monitor that can constantly monitor the change of waistline, so as to solve the problem that people are easily getting obese unconsciously.

According to an embodiment of the present invention, a health monitor includes a belt, a belt-buckle and a reader. The belt-buckle is connected to an end of the belt, and is configured to buckle up with the belt when the belt is rounded in a form of a ring. The reader is located at the belt-buckle, and is configured to read a buckling position where the belt-buckle and the belt buckle up.

In one or more embodiments of the present invention, the health monitor further includes a plurality of markings. The markings are aligned along a belt surface of the belt, and are configured for the reader to read.

In one or more embodiments of the present invention, the reader is an image sensor.

In one or more embodiments of the present invention, the health monitor further includes a transmission unit. The transmission unit is located at the belt-buckle, and is configured to transmit the buckling position read by the reader to a remote device.

In one or more embodiments of the present invention, the transmission unit is a wireless network transmitter or a bluetooth transmitter.

In one or more embodiments of the present invention, the health monitor further includes a battery accommodation slot. The battery accommodation slot is located at the belt-buckle, and is configured for the accommodation of at least a battery therein, in order to provide electric power to the reader.

In one or more embodiments of the present invention, the health monitor further includes an alarm unit. The alarm unit is located at the belt-buckle, and is configured to emit an alert message when the buckling position read by the reader reaches a predetermined position.

In one or more embodiments of the present invention, the alarm unit is a vibrator, a speaker or a warning light.

In one or more embodiments of the present invention, the health monitor further includes a recording unit. The recording unit is located at the belt-buckle, and is configured to record a sound.

In one or more embodiments of the present invention, the health monitor further includes a display unit. The display unit is located at the belt-buckle, and is configured to display the buckling position read by the reader.

When compared with prior art, the embodiments of the present invention have at least the following advantages:

(1) In the embodiment of the present invention, the health monitor is fastened on the waistline of a user in the form of a belt plus a belt-buckle, such that the process of monitoring the waistline change can be carried out in every moment. This allows the user to obtain more detailed and more complete monitoring data.

(2) Through the transmission unit in the belt-buckle, the monitoring data read by the reader can be transmitted at any moment to a remote device, such as a smart phone or a tablet computer. This allows the user to monitor more conveniently the data of the waistline change.

### BRIEF DESCRIPTION OF THE DRAWINGS

The invention can be more fully understood by reading the following detailed description of the embodiment, with reference made to the accompanying drawings as follows:

FIG. 1 is a front view of the health monitor according to one embodiment of the present invention;

FIG. 2 is a 3-dimensional drawing of the health monitor of FIG. 1, wherein the belt is rounded in a form of a ring and is buckled up with the belt-buckle; and

FIG. 3 is a function block diagram of the belt-buckle of the health monitor of FIG. 1.

### DETAILED DESCRIPTION

Drawings will be used below to disclose a plurality of embodiments of the present invention. For the sake of clear illustration, many practical details will be explained together in the description below. However, it is appreciated that, the practical details should not be used to limit the present invention. That is, in some embodiments of the present invention, the practical details are not essential. Moreover, for the sake of drawing simplification, some customary structures and elements in the drawings will be schematically shown in a simplified way. Wherever possible, the same reference numbers are used in the drawings and the description to refer to the same or like parts.

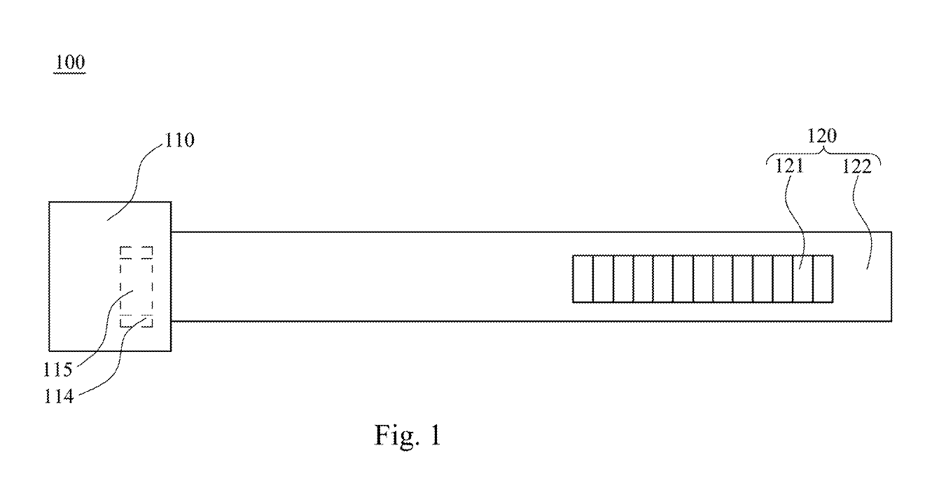

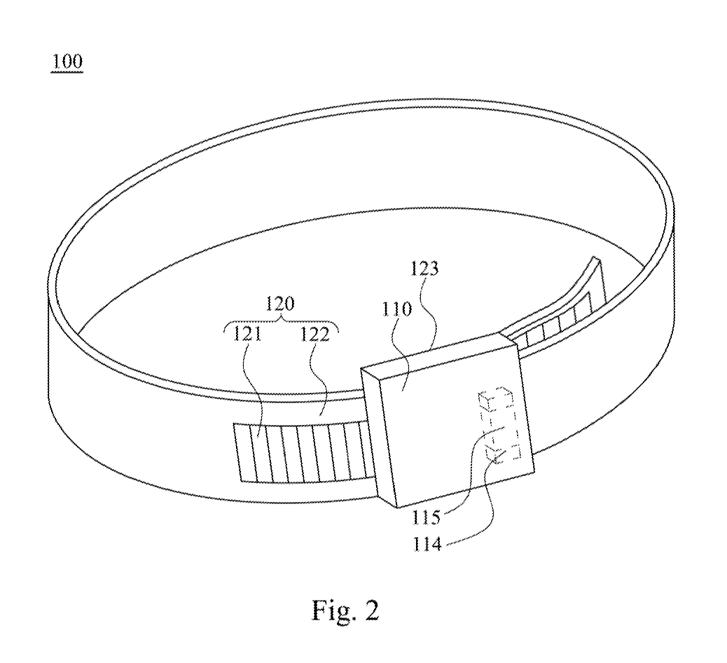

FIG. 1 is a front view of the health monitor 100 according to one embodiment of the present invention. FIG. 2 is a 3-dimensional drawing of the health monitor 100 of FIG. 1, wherein the belt 120 is rounded in a form of a ring and is buckled up with the belt-buckle 110. As shown in FIGS. 1˜2, a health monitor 100 includes a belt 120, a belt-buckle 110 and a reader 111. The belt-buckle 110 is connected to an end of the belt 120, and is configured to buckle up with the belt 120 when the belt 120 is rounded in a form of a ring. The reader 111 is located at the belt-buckle 110, and is configured to read a buckling position 123 where the belt-buckle 110 and the belt 120 buckle up.

In this embodiment, as shown in FIGS. 1˜2, the health monitor 100 further includes a battery accommodation slot 114. The battery accommodation slot 114 is located at the belt-buckle 100, and is configured for the accommodation of at least a battery 115 therein, in order to provide electric power to the reader 111.

In this embodiment, the belt 120 can be of an infinite adjustment or a finite adjustment. For the belt 120 of a finite adjustment, its buckling position 123 with the belt-buckle 110 is limited to the fixing mechanism made in advance on the belt 120. For example, some through holes are made in advance along the belt 120, while a corresponding prong is made on the belt-buckle 110 for passing through one of the through holes. For the belt 120 of an infinite adjustment, its buckling position 123 where the belt-buckle 110 buckles up can be randomly chosen on the belt 120 at any position along the belt 120.

In order to measure the size of the waistline, a belt surface 122 of the belt 120 includes a plurality of markings 121. The markings 121 are aligned along the belt surface 122 of the belt 121, and are configured for the reader 111 to read the buckling position 123 when the belt-buckle 110 and the belt 120 buckle up. The markings 121 can be a notation preset, such as a barcode or Braille, etc. Each of the notations of the markings 121 is different, so as to facilitate the identification and the distinguishing of the markings 121 at different positions. Meanwhile, the reader 111 can be an image sensor.

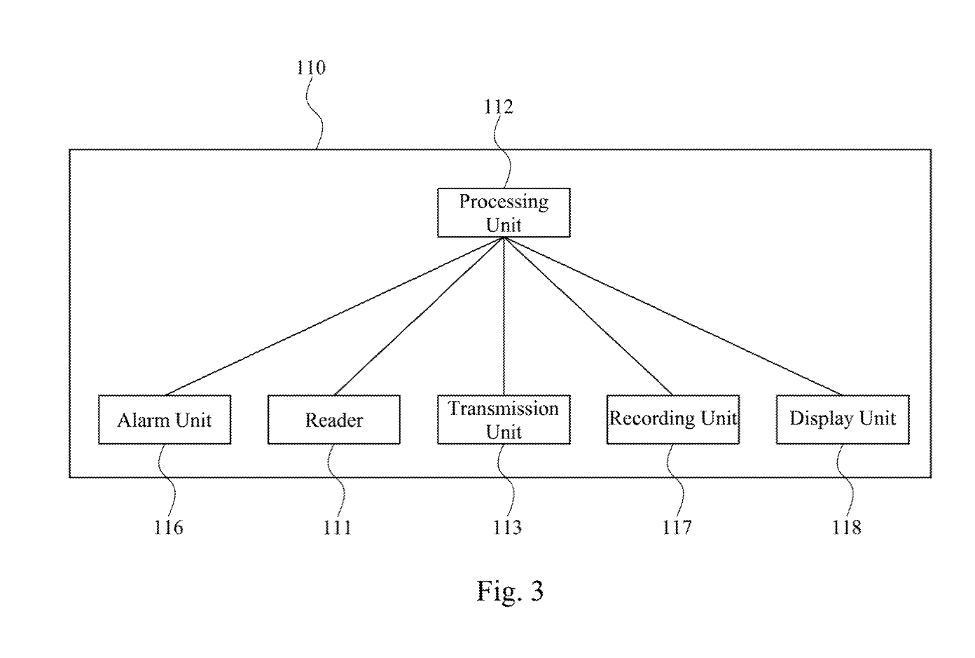

FIG. 3 is a function block diagram of the belt-buckle 110 of the health monitor 100 of FIG. 1. As shown in FIG. 3, the reader 111 is connected to a processing unit 112. The processing unit 112 can perform data conversion and interpretation to the data read by the reader 111, and a corresponding response can be carried out according to the setting preset.

As shown in FIG. 3, the processing unit 112 is further connected to a transmission unit 113. The transmission unit 113 is also located at the belt-buckle 110, and is configured to transmit the buckling position 123 read by the reader 111 to a remote device, after the conversion and interpretation of the processing unit 112. This allows the user to monitor more conveniently the data of the waistline change. Meanwhile, the transmission unit 113 can be a wireless network transmitter or a bluetooth transmitter, and the remote device can be a smart phone or a tablet computer.

In other words, when the user fastens the health monitor 100 at the waistline, with the change of the waistline, the relation positions of the reader 111 and the markings 121 change. The reader 111 then reads different markings 121. After the conversion of the data read by reader 111, such as the length of waistline, the processing unit 112 will transmit the data through the transmission unit 113 to the remote device. In this way, the user can find out the change of the waistline.

The user can also draw a chart using the data of the waistline change. From the changing curve as seen on the chart, the user can easily observe the waistline change in the long-term, medium-term and short-term. For example, the user can observe the waistline change every month, every week and everyday, in order to carry out a more comprehensive health monitoring.

In order to allow the user to conveniently find out the change of waistline even if the user does not bring along the remote device, as shown in FIG. 3, the health monitor 100 further includes a display unit 118. The display unit 118 is located at the belt-buckle 110, and is configured to display the buckling position 123 read by the reader 111, or the length of waistline converted by the processing unit 112. In this way, the user can directly obtain the buckling position 123 read by the reader 111 from the display unit 118, or the waistline length converted by the processing unit 112.

In order to further facilitate the user to directly set the processing unit 112, an input unit can be optionally added to the health monitor 100, allowing the user to directly set the processing unit 112 on the belt-buckle 110.

Apart from measuring the change of human waistline, the health monitor 100 can also carry out health monitor to the user in different patterns. In this embodiment, as shown in FIG. 3, the health monitor 100 further includes a recording unit 117. The recording unit 117 is located at the belt-buckle 110, and is configured to record a sound. In using the health monitor 100, the user fastens the health monitor 100 at the waistline, and the belt-buckle 110 locates probably in front of the abdomen. Thus, the recording unit 117 can receive and record the sound of intestinal peristalsis in the human body. Information such as the frequency of the sound recorded, the volume of the sound, and the time of appearance, can be provided to the doctor for reference to assess the conditions of the intestinal peristalsis, as another reference source for health monitoring.

On the other hand, the reader 111 can assess the frequency and the duration of the user going to the toilet by recording the time of release and buckle up again of the belt-buckle 110 and the belt 120. The relevant data can be tabulated and then analyzed. For example, the frequency of the user going to the toilet each day, the duration of each time, and the time length between each occasion, can all be the reference data for health monitoring.

Apart from carrying out health monitoring, the health monitor 100 can also monitor the food consumption of the user. As shown in FIG. 3, the health monitor 100 further includes an alarm unit 116. The alarm unit 116 is located at the belt-buckle 110, and is configured to emit an alert message under the control of the processing unit 112, when the buckling position 123 read by the reader 111 reaches a predetermined position. For example, the predetermined position of the reader 111 is set to be 85 cm. When the user eats, the processing unit 112 will respond immediately once the waistline reaches 85 cm, and control the alarm unit 116 to emit an alert message. The way to set the predetermined position can be input directly through the input unit of the belt-buckle 110, or set by the setup program of the remote device, and then sending the information of the predetermined position to the transmission unit 113 after the setting is completed. In this embodiment, the alarm unit 116 can be a vibrator, a speaker or a warning light.

Although the present invention has been described in considerable detail with reference to certain embodiments thereof, other embodiments are possible. Therefore, their spirit and scope of the appended claims should not be limited to the description of the embodiments contained herein.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

What is claimed is:

1. A health monitor, comprising:
a belt;
a belt-buckle, connected to an end of the belt, configured to buckle up with the belt when the belt is rounded in a form of a ring; and
a reader, located at the belt-buckle, configured to read a buckling position where the belt-buckle and the belt buckle up.

2. The health monitor of claim 1, further comprising:
a plurality of markings, aligned along a belt surface of the belt, configured for the reader to read.

3. The health monitor of claim 1, wherein the reader is an image sensor.

4. The health monitor of claim 1, further comprising:
a transmission unit, located at the belt-buckle, configured to transmit the buckling position read by the reader to a remote device.

5. The health monitor of claim 4, wherein the transmission unit is a wireless network transmitter or a bluetooth transmitter.

6. The health monitor of claim 1, further comprising:
a battery accommodation slot, located at the belt-buckle, configured for the accommodation of at least a battery therein, in order to provide electric power to the reader.

7. The health monitor of claim 1, further comprising:
an alarm unit, located at the belt-buckle, configured to emit an alert message when the buckling position read by the reader reaches a predetermined position.

8. The health monitor of claim 7, wherein the alarm unit is a vibrator, a speaker or a warning light.

9. The health monitor of claim 1, further comprising:
a recording unit, located at the belt-buckle, configured to record a sound.

10. The health monitor of claim 1, further comprising:
a display unit, located at the belt-buckle, configured to display the buckling position read by the reader.
